(19) United States
(12) Patent Application Publication
(10) Pub. No.: US 2022/0000094 A1
(43) Pub. Date: Jan. 6, 2022

(54) METHODS FOR THE PRESERVATION OF REAGENT RED BLOOD CELLS USING CARBON MONOXIDE

(71) Applicant: Hemanext Inc., Lexington, MA (US)

(72) Inventor: Tatsuro Yoshida, West Newton, MA (US)

(73) Assignee: Hemanext Inc., Lexington, MA (US)

(21) Appl. No.: 17/292,355

(22) PCT Filed: Nov. 18, 2019
(86) PCT No.: PCT/US2019/062020

§ 371 Date: May 7, 2021

Related U.S. Application Data
(60) Provisional application No. 62/769,367, filed on Nov. 19, 2018.

(51) Int. Cl. A01N 1/02; G01N 33/80

(52) U.S. Cl. CPC A01N 1/021; G01N 33/80

(57) ABSTRACT

This application provides methods, compositions, and kits for use blood group determination and the preparation of improved red blood cell containing reagents for use in blood typing of blood prior to its use in transfusion medicine.

### CROSS-REFERENCE TO RELATED APPLICATION

This application claims priority from U.S. Provisional Patent Application Ser. No. 62/769,367, filed Nov. 19, 2018, hereby incorporated by reference in its entirety.

### FIELD OF THE INVENTION

This invention relates to the field of blood group determination and the preparation of improved red blood cell containing reagents for use in blood typing of blood prior to its use in transfusion medicine.

### BACKGROUND OF THE INVENTION

Red Blood Cell (RBC) reagents are manufactured from blood collected from established donors with well-characterized phenotype. RBCs are diluted and packaged in diluent solution to be used as standards for determining blood type of processed RBCs at blood centers/blood banks. In most cases, automated devices are employed for the blood type determinations and for characterizing other important RBC quality parameters. To preserve the surface antigens, RBCs are not fixed and are therefore labile due to the accumulation of storage lesions. These time dependent changes limit the shelf life of these important RBC preparations to about 9 weeks (63 days).

RBC evolved to provide transport oxygen throughout the body, where except for the surface, diffusion from ambient air cannot be relied. RBCs accomplish this function by packing very high concentrations of hemoglobin containing oxidizable ferrous iron in the cytosol. During circulation for approximately 120 days, RBCs are maintained to transport oxygen by elaborate network of metabolic/redox enzymes. However, these elaborate evolutional optimizations are no longer operable once RBCs are removed from circulation and stored hypothermically, resulting in storage-induced damage (storage lesions) that accumulates over the shelf life of stored RBC. One of two major driving forces for development of storage lesions is oxidative damage that was known but not addressed systemically until recently.

Chemical oxidation of iron in hemoglobin is the central reaction that initiates oxidative stress in stored RBCs, the major element for the development of the storage lesion. RBCs contain high concentrations of reactive ferrous iron in the prosthetic group of hemoglobin together with a high concentration of dissolved oxygen. Four iron moieties (ferrous state) in hemoglobin react chemically with oxygen to form methemoglobin (ferric state). As a byproduct, superoxide anion is generated, which is converted by superoxide dismutase to form H₂O₂, a major reactive oxygen species (ROS) and a substrate for hydroxyl radical (OH.) generation. In vivo, methemoglobin is reduced back to hemoglobin by reductase enzymes but these enzyme activities are curtailed under hypothermic storage conditions. Coupled with higher dissolved oxygen concentrations stemming from increased solubility at low temperature, this phenomenon results in enhanced production of methemoglobin and superoxide anion. ROS molecules react with lipids and structural proteins in RBC damaging integrity and reducing in vivo circulation life. ROS also attack critical enzymes or surface molecules that makes ‘product’ RBCs valuable, diminishing efficacy or utility.

Hemoglobin's affinity toward CO is about 400 times higher than O₂, and CO does not readily react chemically with heme. The heme-CO complex is very stable, and thus greatly reduces oxidative RBC storage lesions as hemoglobin oxidation by oxygen is the main driver of oxidative stress during hypothermic RBC storage. Unlike O₂, CO does not react readily with ferrous iron however it prevents Hb oxidation by stabilizing it as Hb-CO, and greatly reduces oxidative storage lesion development in hypothermically (i.e., 1-6° C.) stored RBCs. ROS damage can be further reduced by storing the RBCs under oxygen free conditions, either under CO or an inert gas like nitrogen.

Although high affinity binding of CO to Hb renders Hb and RBCs containing Hb-CO useless as an oxygen carrier until the CO is released, the stabilization of Hb by CO is beneficial during storage and can extend the shelf life of the Reagent RBCs not only prior to being opened or reconstituted for use, but also after opening. Much more than RBCs for transfusions, the Reagent RBCs are highly characterized and carefully controlled reagents of great value. Even small improvements to the shelf life can significantly reduce costs for blood banking operations. CO-treatment of reagent RBCs reduces storage lesion accumulation and prolongs the shelf life.

### SUMMARY OF THE INVENTION

The present disclosure provides for, and includes, a method for preserving reagent red blood cells (RBC) comprising obtaining packed red blood cells, flushing the packed red blood cells with a gas comprising carbon monoxide to prepare carbon monoxide saturated RBCs (CO-Hb RBCs) and storing the CO-Hb RBCs under anaerobic conditions in the presence of carbon monoxide (CO), wherein surface antigens of said CO-Hb RBCs are stabilized.

The present disclosure provides for, and includes, kits comprising one or more vials of carbon monoxide saturated RBCs (CO-Hb RBCs) having a plurality of CO-Hb RBCs having a common set of surface antigens in a buffer and instructions for use

The present disclosure provides for, and includes, a vial of carbon monoxide saturated RBCs (CO-Hb RBCs) comprising a buffer and CO-Hb RBCs selected from the group consisting of :CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s; CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a}; CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w}; CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b}; CO-Hb RBCs are type-O cells that are positive for the Rh antigens D, C, and e; CO-Hb RBCs are type-O cells that are positive for the Rh antigens D, C, and e, I, Lu^{b}, Js^{b}, Kp^{b}, and Yt^{a}; CO-Hb RBCs are type-O cells that are positive for the Rh antigens D, C, and e, I, Lu^{b}, Js^{b}, Kp^{b}, and Yt^{a,} and that are negative for the surface antigens Js^{a}, Kp^{a}, Wr^{a}, Di^{a} V^{w}, V, Lu^{a} and C^{w}; CO-Hb RBCs are type-A cells that are positive for the surface antigen A1; CO-Hb RBCs are type-A cells that are positive for the surface antigen A1 and are negative for surface antigens D, C, and E;CO-Hb RBCs are type-A cells that are positive for the surface antigen A2; CO-Hb RBCs are type-A cells that are positive for the surface antigen A2; and are negative for surface antigens D, C, and E; CO-Hb RBCs are type-B cells that are positive for the surface antigen B; CO-Hb RBCs are type-B cells that are positive for the surface antigen B and are negative for surface antigens D, C, and E; CO-Hb RBCs are type-O cells that are positive for the surface antigens D, C, and e and having the Rh phenotype R₁R₁; CO-Hb RBCs are type-O cells that are positive for the surface antigens D, C^{w}, and e and having the Rh phenotype R₁^{w}R₁; CO-Hb RBCs are type-O cells that are positive for the surface antigens D, c, and E and having the Rh phenotype R₂R₂; CO-Hb RBCs are type-O cells that are positive for the surface antigens d, c, and e and having the Rh phenotype rr; CO-Hb RBCs are type-O cells that are positive for the surface antigens D, C, and e and having the Rh phenotype R₁R₁ and are positive for the surface antigens Lu^{b}, Js^{b}, Kp^{b}, and Yt^{a}; CO-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a}; CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a}; CO-Hb RBCs are type-O cells that are positive for the surface antigens d, c, and e and having the Rh phenotype rr and are positive for the surface antigens Lu^{b}, Js^{b}, Kp^{b}, and Yt^{a}; CO-Hb RBCs are type-O cells that are positive for the surface antigens D, C, and e and having the Rh phenotype R₁R₁ and are negative for the surface antigens Js^{a}, Kp^{a}, Wr^{a}, Di^{a}, Vw, V, Lu^{a} and C^{w}; CO-Hb RBCs are type-O cells that are positive for the surface antigens D, C^{w}, and e and having the Rh phenotype R₁^{w}R₁ and are negative for the surface antigens Js^{a,} Kp^{a}, Wr^{a}, Di^{a}, Vw, V, Lu^{a} and C^{w}; CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, Vw, V, Lu^{a} and C^{w}; CO-Hb RBCs are type-O cells that are positive for the surface antigens d, c, and e and having the Rh phenotype rr and are negative for the surface antigens Js^{a}, Kp^{a}, Wr^{a}, Di^{a}Vw, V, Lu^{a} and C^{w}; CO-Hb RBCs are type-O cells that are positive for the surface antigens D, C, and e and having the Rh haplotype R₁ or are positive for the surface antigens D, c, and e and having the Rh haplotype R₂; CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};CO-Hb RBCs are type-O cells that are positive for the surface antigens D, C, and e and having the Rh haplotype R₁ and are negative for the surface antigens Js^{a}, Kp^{a}, Wr^{a}, Di^{a}, Vw, V, Lu^{a} and C^{w} or are positive for the surface antigens D, c, and e and having the Rh haplotype R₂ and are negative for the surface antigens Js^{a}, Kp^{a}, Wr^{a}, Di^{a}, Vw, V, Lu^{a} and C^{w}; CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a}; CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens; CO-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a}; CO-Hb RBCs are type-O cells that are positive for binding of antibodies to the D antigen and said cells are sensitized with anti-D(Rh₀) serum; CO-Hb RBCs are type-A cells that are positive for binding of antibodies to the A₂ antigen; CO-Hb RBCs are type-B cells that are positive for binding of antibodies to the B antigen and are negative for binding of anti-D(Rh₀) antibodies; CO-Hb RBCs are type-A cells that are positive for binding of antibodies to the A₁ antigen and are negative for binding of anti-D(Rh₀) antibodies; CO-Hb RBCs are type-AB cells that are positive for binding of antibodies to the A₁, B antigens and the Rh antigens d, c, and e of Rh blood group rr (dce/dec); CO-Hb RBCs are type-O cells that are positive for binding of antibodies to the Rh antigens D, d, C, c, and e and having the Rh phenotype R₁r (DCe/dce); CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b}; CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, Vw, V, Lu^{a} and C^{w} antigens.

### BRIEF DESCRIPTION OF THE DRAWINGS

FIG. 1 is a graph showing the deoxygenation and carbon monoxidization of a red cell concentrate in an embodiment according to Example 2(a).

### DETAILED DESCRIPTION OF THE INVENTION

Blood group serology requires the determination of blood cell compatibility between a blood donor and a patient recipient before a transfusion or organ transplant involving the patient. Blood cell compatibility is determined by the non-occurrence of an immunological reaction between antibodies contained in the blood serum of a patient and antigens present on blood cells from the donor. Tests for blood cell typing and compatibility are generally of two types: (1) agglutination tests which determine whether a specific antibody added to the cells will cause their agglutination, and (2) cell lysis tests which determine whether a specific antibody added to the tested cells together with serum complement results in hemolysis. In blood cell typing and compatibility test procedures commonly used, both agglutination and cell lysis tests are carried out either manually by a trained technician or using automated devices. The presence of an immunological reaction is incompatible with transfusion and transplantation therapies.

The International Society of Blood Transfusion lists 33 blood group systems representing over 300 antigen. See Lögdberg et al., “Human blood group genes 2004: Chromosomal locations and cloning strategies,” Transfus. Med. Rev. 19:45-57 (2005) and Lögdberg et al., “Human blood group genes 2010: Chromosomal locations and cloning strategies revisited,” Transfus. Med. Rev. 25:36-46 (2011). Cloning and sequencing demonstrates that the genes of these blood group systems are autosomal, except XG and XK which are X-borne, and MIC2 which is present on both X and Y chromosomes. Many different blood group antigens are found on the surface of the red blood cells of every individual. These antigens, the products of inherited genes, exist in combinations that are likely to be unique between all individuals except identical twins.

A number of significant blood group systems are well known in the art and are presented in Table 1.

TABLE 1
Common Blood Groups

| Name | Symbol | Antigen # | Gene Name | Chromosome |
|---|---|---|---|---|
| ABO | ABO | 5 | ABO | 9 |
| MNS | MNS | 43 | GYPA, GYPB, GYPE | 4 |
| P | P1 | 1 | P1 | 22 |
| Rhesus | Rh | 49 | RhD, RhCE | 1 |
| Lutheran | LU | 20 | LU | 19 |
| Kell | KEL | 25 | KEL | 7 |
| Lewis | LE | 6 | FUT3 | 19 |
| Duffy | FY | 6 | FY | 1 |
| Kidd | Jk | 3 | SLC14A1 | 18 |

www.ncbi.nlm.nih.gov/pmc/articles/PMC4260296/

Blood grouping is generally the process of testing red cells to determine which antigens are present and which are absent, normally utilizing antibodies to the antigen tested for. Additionally, when a person does not have a particular red cell antigen on his or her red blood cells, his or her serum may contain an antibody to that antigen. Whether or not the antibody is present in the serum depends on whether the person's immune system has been previously challenged by, and responded to, that specific antigen or something very similar to it. For example, a person whose red blood cells are Type A, i.e., having “A” antigens on the red cells, will have anti-B antibodies in his or her serum. Thus, if such a person is given type B blood, an immunological reaction will occur with possible serious clinical consequences.

Before transfusion therapy, the collected blood is tested for compatibility by analyzing the blood groups. Testing of blood groups is carried out by testing RBCs for the various antigens (A, B, etc.) and testing the serum for antibodies to the antigens. For the former test, RBCs from the sample blood is incubated with antibodies that recognize each of the blood group antigens and scored for binding either using aggregation or other known immunological approach. Testing of the serum can be performed in a variety of ways such as by ELISA where the antigen is provided and antibody binding is detected indirectly through an enzyme or fluorescent reporter. A more traditional approach is to employ RBCs previously characterized as expressing specific antigens in agglutination assays called hemagglutination assays. In short, the agglutination assay comprises mixing reagent RBCs together with serum or plasma from the test sample of blood. Antibodies in the test sample, typically IgM having five antigen binding sites cross-link cells together causing clumping that can be seen macroscopically. Divalent IgG antibodies are also suitable for agglutination assays. Agglutination assays, including those directed to blood typing are well known in the art. See Löw et al., “Antiglobulin test in low-ionic strength salt solution for rapid antibody screening and cross-matching,” Vox Sang 26:53-61 (1974); Malyska et al., “The gel test,” Laboratory Medicine 25:81 (1994); and Technical manual. 14th ed. Bethesda, Md.: American Association of Blood Banks, 2002.

Among the more common Reagent RBCs typically used for reverse typing have on their surface either A, A, B or no ABO antigens (Type A1, Type A2, Type B, Type O). These cells are useful for detecting preformed antibodies which will cause agglutination of the reagent RBCs. For forward type testing, monoclonal anti-A and anti-B are used to detect the presence of their respective antigen on a sample red cell surface. Another well-known blood group is the Rh blood group having 58 different antigenic types, though nine types are the most common. The blood groups and the designations of the antigens are presented in Table 2 and Table 3. Byrne et al., “Review: other blood group systems—Diego, Yt, Xg, Scianna, Dombrock, Colton, Landsteiner-Wiener, and Indian,” Immunohematology 20(1):50-8 (2004); Daniels, “The molecular genetics of blood group polymorphism,” Transpl. Immunol. 14(3-4):143-53 (2005); Daniels, “Functions of red cell surface proteins,” Vox Sang. 93(4):331-40 (2007); Eyler et al., “The Lutheran glycoprotein: a multifunctional adhesion receptor.” Transfusion 46(4):668-77 (2006); Palacajornsuk, “Review: molecular basis of MNS blood group variants,” Immunohematology 22(4):171-82 (2006); Quill, “Medicine. Blood-matching goes genetic,” Science 14;319(5869):1478-9 (2008) ; Westhoff, “The structure and function of the Rh antigen complex,” Semin Hematol 44(1):42-50 (2007); Westhoff, “Review: the Kell, Duffy, and Kidd blood group systems,” Immunohematology 20(1):37-49 (2004); and Yamamoto, “Review: ABO blood group system—ABH oligosaccharide antigens, anti-A and anti-B, A and B glycosyltransferases, and ABO genes,” Immunohematology 20(1):3-22 (2004), each of which are hereby incorporated by reference in their entireties.

TABLE 2
Blood Groups MNS, RH, LU, KEL and DI

| | 1 | 2 | 4 | 5 | 6 | 10 |
|---|---|---|---|---|---|---|
| 1 | ABO | MNS | RH | LU | KEL | DI |
| 2 | A | M | D | Lu^{a} | K | Di^{a} |
| 3 | B | N | C | Lu^{b} | k | Di^{b} |
| 4 | A, B | S | E | Lu3 | Kp^{a} | Wr^{a} |
| 5 | A1 | s | c | Lu4 | Kp^{b} | Wr^{b} |
| 6 | | U | e | Lu5 | Ku | Wd^{a} |
| 7 | | He | f | Lu6 | Js^{a} | Rb^{a} |
| 8 | | Mi^{a} | Ce | Lu7 | Js^{b} | WARR |
| 9 | | M^{c} | C^{w} | Lu8 | . . . | ELO |
| 10 | | Vw | c^{x} | Lu9 | . . . | Wu |
| 11 | | Mur | V | . . . | Ul^{a} | Bp^{a} |
| 12 | | M^{g} | E^{w} | Lu11 | K11 | Mo^{a} |
| 13 | | Vr | G | Lu12 | K12 | Hg^{a} |
| 14 | | M^{e} | . . . | Lu13 | K13 | Vg^{a} |
| 15 | | Mt^{a} | . . . | Lu14 | K14 | Sw^{a} |
| 16 | | St^{a} | . . . | . . . | . . . | BOW |
| 17 | | Ri^{a} | . . . | Lu16 | K16 | NFLD |
| 18 | | Cl^{a} | Hrₒ | Lu17 | K17 | Jn^{a} |
| 19 | | Ny^{a} | Hr | Au^{a} | K18 | KREP |
| 20 | | Hut | hr^{S} | Au^{b} | K19 | Tr^{a} |
| 21 | | Hil | VS | Lu20 | Km | Fr^{a} |
| 22 | | M^{v} | C^{G} | Lu21 | Kp^{c} | SW1 |
| 23 | | Far | CE | | K22 | |
| 24 | | s^{D} | D^{w} | | K23 | |
| 25 | | Mit | . . . | | K24 | |
| 26 | | Dantu | . . . | | VLAN | |
| 27 | | Hop | c-like | | TOU | |
| 28 | | Nob | cE | | RAZ | |
| 29 | | En^{a} | hr^{H} | | VONG | |
| 30 | | En^{a}KT | Rh29 | | KALT | |
| 31 | | ‘N’ | Go^{a} | | KTIM | |
| 32 | | Or | hr^{B} | | KYO | |
| 33 | | DANE | Rh32 | | KUCI | |
| 34 | | TSEN | Rh33 | | KANT | |
| 35 | | MINY | Hr^{B} | | KASH | |
| 36 | | MUT | Rh35 | | | |
| 37 | | SAT | Be^{a} | | | |
| 38 | | ERIK | Evans | | | |
| 39 | | Os^{a} | . . . | | | |
| 40 | | ENEP | Rh39 | | | |
| 41 | | ENEH | Tar | | | |
| 42 | | HAG | Rh41 | | | |
| 43 | | ENAV | Rh42 | | | |
| 44 | | MARS | Crawford | | | |
| 45 | | ENDA | Nou | | | |
| 46 | | ENEV | Riv | | | |
| 47 | | MNTD | Sec | | | |
| 48 | | | Dav | | | |
| 49 | | | JAL | | | |
| 50 | | | STEM | | | |
| 51 | | | FPTT | | | |
| 52 | | | MAR | | | |
| 53 | | | BARC | | | |
| 54 | | | JAHK | | | |
| 55 | | | DAK | | | |
| 56 | | | LOCR | | | |
| 57 | | | CENR | | | |
| 58 | | | CEST | | | |

TABLE 3
Blood Group Antigens

| | | Antigen Number | | | | | | | |
|---|---|---|---|---|---|---|---|---|---|
| System | | 1 | 2 | 3 | 4 | 5 | 6 | 7 | 8 |
| 3 | P | P1 | . . . | . . . | | | | | |
| 7 | LE | Le^{b} | Le^{b} | Le^{ab} | Le^{bH} | ALe^{b} | BLe^{b} | | |
| 8 | FY | Fy^{a} | Fy^{b} | Fy3 | Fy4 | Fy5 | Fy6 | | |
| 9 | JK | Jk^{a} | Jk^{b} | Jk3 | | | | | |
| 11 | YT | Yt^{a} | Yt^{b} | | | | | | |
| 12 | XG | Xg^{a} | CD99 | | | | | | |
| 13 | SC | Sc1 | Sc2 | Sc3 | Rd | STAR | SCER | SCAN | |
| 14 | DO | Do^{a} | Do^{b} | Gy^{a} | Hy | Jo^{a} | DOYA | | |
| 15 | CO | Co^{a} | Co^{b} | Co3 | | | | | |
| 16 | LW | . . . | . . . | . . . | . . . | LW^{a} | LW^{ab} | LW^{b} | |
| 17 | CH/RG | Ch1 | Ch2 | Ch3 | Ch4 | Ch5 | Ch6 | WH | |
| 18 | H | H | | | | | | | |
| 19 | XK | Kx | | | | | | | |
| 20 | GE | . . . | Ge2 | Ge3 | Ge4 | Wb | Ls^{a} | An^{a} | Dh^{a} |
| 21 | CROM | Cr^{a} | Tc^{a} | Tc^{b} | Tc^{c} | Dr^{a} | Es^{a} | IFC | WES^{a} |
| 22 | KN | Kn^{a} | Kn^{b} | McC^{a} | Sl1 | Yk^{a} | McC^{b} | Sl2 | Sl3 |
| 23 | IN | In^{a} | In^{b} | INFI | INJA | | | | |
| 24 | OK | Ok^{a} | | | | | | | |
| 25 | RAPH | MER2 | | | | | | | |
| 26 | JMH | JMH | JMHK | JMHL | JMHG | JMHM | | | |
| 27 | I | I | | | | | | | |
| 28 | GLOB | P | | | | | | | |
| 29 | GIL | GIL | | | | | | | |
| 30 | RHAG | Duclos | Ol^{a} | Duclos- | | | | | |
| | | | | like | | | | | |

| | | Antigen Number | | | | | | |
|---|---|---|---|---|---|---|---|---|
| System | | 9 | 10 | 11 | 12 | 13 | 14 | 15 |
| 3 | P | | | | | | | |
| 7 | LE | | | | | | | |
| 8 | FY | | | | | | | |
| 9 | JK | | | | | | | |
| 11 | YT | | | | | | | |
| 12 | XG | | | | | | | |
| 13 | SC | | | | | | | |
| 14 | DO | | | | | | | |
| 15 | CO | | | | | | | |
| 16 | LW | | | | | | | |
| 17 | CH/RG | | | Rg1 | Rg2 | | | |
| 18 | H | | | | | | | |
| 19 | XK | | | | | | | |
| 20 | GE | GEIS | | | | | | |
| 21 | CROM | WES^{b} | UMC | GUTI | SERF | ZEN A | CROV | CRAM |
| 22 | KN | KCAM | | | | | | |
| 23 | IN | | | | | | | |
| 24 | OK | | | | | | | |
| 25 | RAPH | | | | | | | |
| 26 | IM11 | | | | | | | |
| 27 | I | | | | | | | |
| 28 | GLOB | | | | | | | |
| 29 | GIL | | | | | | | |
| 30 | RHAG | | | | | | | |

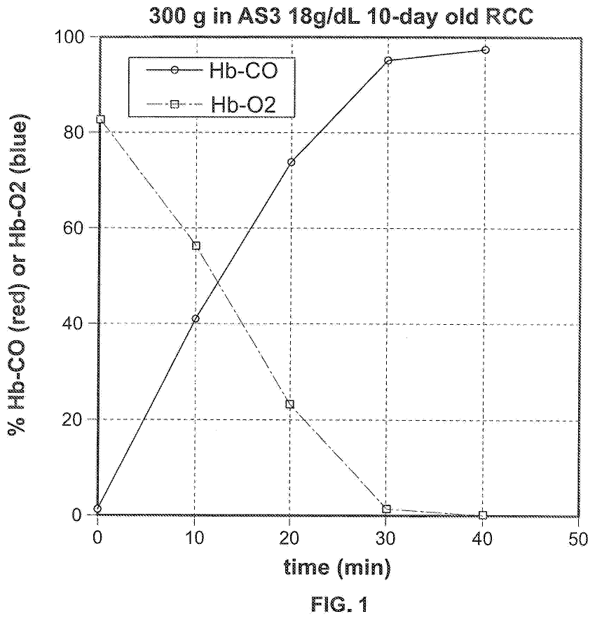

The present disclosure provides for, and includes, methods to reduce degradation of blood group antigens during storage. More specifically, the present disclosure provides for reducing the formation of storage lesions in RBCs during storage including, but not limited to, ROS induced lesions. In brief, RBCs are collected exposed to an atmosphere of carbon monoxide (CO) for a period of time sufficient to remove oxygen (O₂) bound to the heme group of hemoglobin. As shown in Example 2 and FIG. 1, the exchange of oxygen for CO occurs rapidly and essentially complete in 30 minutes and three exchanges of gas. Methods that bring the CO into contact with the blood, particularly those that increase the surface to volume ration of the CO/liquid interface would significantly reduce the length of time necessary to exchange the bound oxygen with carbon monoxide. As discussed above, carbon monoxide has a significantly higher affinity for hemoglobin that oxygen (e.g., 400×), thus exposure of RBCs to CO at any level and time will begin the exchange process and provided sufficient CO, will drive the exchange for completion.

The present disclosure provides for, includes, but is not limited to, exchanging the oxygen for carbon monoxide according the methods shown in Example 2. In an aspect, red cell concentrate (RCC, also known as packed red blood cells) are held in a container and CO is introduced and the container is shaken or mixed gently for a period. In an aspect, the container is a standard blood storage bag comprising polyvinyl chloride. In an aspect, the CO gas is replaced and the mixing repeated one or more time until the hemoglobin is saturated with CO (e.g., Hb-CO RBCs are produced). In another aspect, the container containing the RCCs are provided with a volume of CO and left overnight with, or without, occasional mixing. Mixing improves the rate of exchange, but is not required. In another aspect, the blood and CO is separated by a gas permeable membrane. This can be done using methods known in the art, for example using a Sorin D100 oxygenator and providing CO rather than oxygen. The advantage of a membrane based approach is that the gas can be continuously replaced thereby maintaining the concentration gradient and increasing the rate of exchange.

In another aspect, CO gas can be bubbled through a container or bag of RBCs. Not to be limited by theory, it is thought that by maintaining the bubbles to less than 1 μm in diameter, lysis of the red cells can be prevented or minimized. The ‘bubbling’ method shares the same advantages as the membrane based approach as the CO bubble will provide for a maximal concentration difference thereby facilitating the kinetics of the exchange reaction. The bubble approach also provides for the further advantage of mixing the RBCs.

While it may be preferable to perform carbon monoxide exchange on RCCs, the specification provides for, and includes, exchanging CO for oxygen on blood at any stage of the process. In an aspect, the CO is exchanged on whole blood, prior to removing for example platelets or leukocytes. In an aspect, CO is exchanged on leukoreduced blood. The methods of the present specification can be applied to RBCs prepared by apheresis or collected using traditional methods. The methods may also be performed after processing of the RBCs into a suitable buffer for reagent use and storage. See, for example, U.S. Patent Publication No. 2011/0045455, published Feb. 26, 2001; and International Patent Publication No. WO 1983/003477, published Oct. 13, 1983. Other storage solutions compatible with blood typing methods are known in the art.

The present specification provides for, and includes, methods for preparing CO-Hb RBCs that further includes storing said CO-Hb RBCs under anaerobic conditions in the presence of CO. In an aspect, the cells are prepared as described above and transferred to a vial under an atmosphere comprising carbon monoxide. In another aspect, the CO-Hb RBCs are transferred to a container for storage having an nitrogen atmosphere. As provided herein, during storage, any suitable non-oxygen containing gas is suitable. In certain aspect, additional CO is provided before sealing the container for CO-Hb RBCs storage.

The present disclosure provides for, and includes, a method of preserving reagent red blood cells (RBC) comprising obtaining packed red blood cells that selected from, but not limited to the following 40 immunological types:
1.Blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
2.Blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
3. Blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yta and negative for the surface antigens Js^{a}, Kp^{a}, Wᵣa, Di^{a}, V^{w}, V, Lu^{a}, and C^{w};
4.Blood type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b};
5.Blood type-O cells that are positive for the Rh antigens D, C, and e;
6.Blood type-O cells that are positive for the Rh antigens D, C, and e, I, Lu^{b}, Js^{b}, Kp^{b}, and Yt^{a};
7.Blood type-O cells that are positive for the Rh antigens D, C, and e, I, Lu^{b}, Js^{b}, Kp^{b}, and Yta, and that are negative for the surface antigens Js^{a}, Kp^{a}, Wr^{a}, Di^{a} V^{w}, V, Lu^{a} and C^{w};
8.Blood type-A cells that are positive for the surface antigen A1;
9.Blood type-A cells that are positive for the surface antigen A1 and are negative for surface antigens D, C, and E;
10. Blood type-A cells that are positive for the surface antigen A2;
11. Blood type-A cells that are positive for the surface antigen A2; and are negative for surface antigens D, C, and E;
12. Blood type-B cells that are positive for the surface antigen B;
13. Blood type-B cells that are positive for the surface antigen B and are negative for surface antigens D, C, and E;
14. Blood type-O cells that are positive for the surface antigens D, C, and e and having the Rh phenotype R₁R₁;
15. Blood type-O cells that are positive for the surface antigens D, C^{w}, and e and having the Rh phenotype R₁^{w}R₁;
16. Blood type-O cells that are positive for the surface antigens D, c, and E and having the Rh phenotype R₂R₂;
17. Blood type-O cells that are positive for the surface antigens d, c, and e and having the Rh phenotype rr;
18. Blood type-O cells that are positive for the surface antigens D, C, and e and having the Rh phenotype R₁R₁ and are positive for the surface antigens Lu^{b}, Js ^{b}, Kp^{b}, and Yt^{a};
19. Blood type-O cells that are positive for the surface antigens D, C^{w}, and e and having the Rh phenotype R₁^{w}R₁ and are positive for the surface antigens Lu^{b}, Js_{b}, Kp^{b}, and Yt^{a};
20. Blood type-O cells that are positive for the surface antigens D, c, and E and having the Rh phenotype R₂R₂ and are positive for the surface antigens Lu^{b}, Js^{b}, Kp^{b}, and Yt^{a};
21. Blood type-O cells that are positive for the surface antigens d, c, and e and having the Rh phenotype rr and are positive for the surface antigens Lu^{b}, Js^{b}, Kp^{b}, and Yt^{a};
22. Blood type-O cells that are positive for the surface antigens D, C, and e and having the Rh phenotype R₁R₁ and are negative for the surface antigens Js^{a}, Kp^{a}, Wr^{a}, Di^{a}, Vw, V, Lu^{a} and C^{w};
23. Blood type-O cells that are positive for the surface antigens D, C^{w}, and e and having the Rh phenotype R₁^{w}R₁ and are negative for the surface antigens Js^{a}, Kp^{a}, Wr^{a}, Di^{a}, V_{w}, V, Lu^{a} and C^{w};
24. Blood type-O cells that are positive for the surface antigens D, c, and E and having the Rh phenotype R₂R₂ and are negative for the surface antigens Js^{a}, Kp^{a}, Wr^{a}, Di^{a}, Vw, V, Lu^{a} and C^{w};
25. Blood type-O cells that are positive for the surface antigens d, c, and e and having the Rh phenotype rr and are negative for the surface antigens Js^{a}, Kp^{a}, Wr^{a}, Di^{a}, Vw, V, Lu^{a} and C^{w};
26. Blood type-O cells that are positive for the surface antigens D, C, and e and having the Rh haplotype R₁ or are positive for the surface antigens D, c, and e and having the Rh haplotype R₂;
27. Blood type-O cells that are positive for the surface antigens D, C, and e and having the Rh haplotype Rᵢ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};
28. Blood type-O cells that are positive for the surface antigens D, C, and e and having the Rh haplotype R₁ and are negative for the surface antigens Js^{a}, Kp^{a}, Wr^{a}, Di^{a}, Vw, V, Lu^{a} and C^{w} or are positive for the surface antigens D, c, and e and having the Rh haplotype R₂ and are negative for the surface antigens Js^{a}, Kp^{a}, Wr^{a}, Di^{a}, V_{w}, V, Lu^{a} and C^{w};
29. Blood type-O cells that have been ficin treated and that are negative of the surface antigens M, N, Fy^{a}, Fy^{b}, S, s, Xg^{a}, Pr, Ch^{a}, Rg^{a} and Yk^{a};
30. Blood type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
31. Blood type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
32. Blood type-O cells that are positive for binding of antibodies to the D antigen and said CO-Hb RBCs are sensitized with anti-D(Rh₀) serum;
33. Blood type-A cells that are positive for binding of antibodies to the A₂ antigen;
34. Blood type-B cells that are positive for binding of antibodies to the B antigen and are negative for binding of anti-D(Rh₀) antibodies;
35. CO-Hb RBCs are type-A cells that are positive for binding of antibodies to the A₁ antigen and are negative for binding of anti-D(Rh₀) antibodies;
36. Blood type-AB cells that are positive for binding of antibodies to the A₁, B antigens and the Rh antigens d, c, and e of Rh blood group rr (dce/dec);
37. Blood type-O cells that are positive for binding of antibodies to the Rh antigens D, d, C, c, and e and having the Rh phenotype R₁r (DCe/dce);
38. Blood type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b};
39. Blood type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and
40. Blood type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, Vw, V, Lu^{a} and C^{w} antigens.

As would be understood to a person of ordinary skill in the art, the selection of RBCs suitable for the preparation of reagent RBCs is not limited to the combinations of blood types as provided above. A person or ordinary skill would recognized that RBCs having any antigen combination of the groups recited in Tables 2 and 3 are useful for the methods of the present application. Indeed, a person of ordinary skill in the art would recognize that any RBC, once characterized immunologically would suitable for the preparation of Reagent RBCs preserved with carbon monoxide (Hb-CO RBCs).

The present specification provides for, and includes, exchanging the oxygen bound on hemoglobin with carbon monoxide using any known methods in the art, including but not limted to the methods shown in the examples. Thus, as used herein, the term ‘flushing’ refers to any method that brings carbon monoxide gas into contact with RBCs including bubbling or passing through a membrane.

The present specification provides for, and includes, kits comprising carbon monoxide stabilized red blood cells (Hb-CO RBCs). Kits of the present specification may include one or more of the 40 specific types Hb-CO RBCs of the cells described above, but are not limited to those cells. Other suitable Hb-CO RBCs can be prepared as needed. In certain aspects, the kits provide the cells suspended in a suitable buffer and the preparation of the cells may include various washing steps either before carbon monoxide exchange, or after carbon monoxide exchange. Additional reagents and buffer necessary for performing immunological assays may be included in the kits. In many aspects, the kits will include instructions on the performance of the assays, lot characterization of the Hb-CO RBCs and other materials pertinent to a person of skill in the art. In some aspect, the kits of the present specification may include various labware such as tubes, pipettes, buffers, etc.

The present specification provides for, and includes, containers containing the CO-Hb RBCs described herein. In an aspect, the container is a vial. In another aspect, the container is a tube. In yet another aspect, the container is an ampule.

The present specification provides for, and includes, the following embodiments:

Embodiment 1. A method for preserving reagent red blood cells (RBC) comprising:
a) obtaining packed red blood cells;
b) flushing said packed red blood cells with a gas comprising carbon monoxide to prepare carbon monoxide saturated RBCs (CO-Hb RBCs); and
c) storing said CO-Hb RBCs under anaerobic conditions in the presence of carbon monoxide (CO), wherein surface antigens of said CO-Hb RBCs are stabilized.

Embodiment 2. The method of embodiment 1, wherein said gas does not comprise oxygen.

Embodiment 3. The method of any one of the preceding embodiments, wherein said CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s.

Embodiment 4. The method of any one of the preceding embodiments, wherein said CO-Hb RBCs are blood group O cells that further are positive for the surface antigens I, Lu^{a}, Lu^{b}, Js^{b}, Kp^{b}, and Yt^{a}.

Embodiment 5. The method of any one of the preceding embodiments, wherein said CO-Hb RBCs are blood group O cells that are negative for the surface antigens Js^{a}, Kp^{a}, Wr^{a}, Di^{a}, V^{w}, V, Lu^{a,} and C^{w}.

Embodiment 6. The method of any one of the preceding embodiments, wherein said CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b}.

Embodiment 7. The method of any one of the preceding embodiments, wherein said CO-Hb RBCs are type-O cells that are positive for the Rh antigens D, C, and e.

Embodiment 8. The method of any one of the preceding embodiments, wherein said CO-Hb RBCs are type-O cells that are positive for the surface antigens I, Lu^{b}, Js^{b}, Kp^{b}, and Yt^{a}.

Embodiment 9. The method of any one of the preceding embodiments, wherein said CO-Hb RBCs are type-O cells that are negative for the surface antigens Js^{a}, Kp^{a}, Wr^{a}, Di^{a} V^{w}, V, Lu^{a} and C^{w}.

Embodiment 10. The method of any one of the preceding embodiments, wherein said CO-Hb RBCs are type-A cells that are positive for the surface antigen A1.

Embodiment 11. The method of any one of the preceding embodiments, wherein said CO-Hb RBCs are type-A cells that are negative for surface antigens D, C, and E.

Embodiment 12. The method of any one of the preceding embodiments, wherein said CO-Hb RBCs are type-A cells that are positive for the surface antigen A2.

Embodiment 13. The method of any one of the preceding embodiments, wherein said CO-Hb RBCs are type-A cells that are negative for surface antigens D, C, and E.

Embodiment 14. The method of any one of the preceding embodiments, wherein said CO-Hb RBCs are type-B cells that are positive for the surface antigen B.

Embodiment 15. The method of any one of the preceding embodiments, wherein said CO-Hb RBCs are type-B cells that are negative for surface antigens D, C, and E.

Embodiment 16. The method of any one of the preceding embodiments, wherein said CO-Hb RBCs are type-O cells that
are positive for the surface antigens D, C, and e and having the Rh phenotype R₁R₁;
are positive for the surface antigens D, C^{w}, and e and having the Rh phenotype R₁^{w}R₁;
are positive for the surface antigens D, c, and E and having the Rh phenotype R₂R_{2;} or
are positive for the surface antigens d, c, and e and having the Rh phenotype rr.

Embodiment 17. The method of any one of the preceding embodiments, wherein said CO-Hb RBCs are type-O cells that are positive for the surface antigens Lu^{b}, Js^{b}, Kp^{b}, and Yt^{a}.

Embodiment 18. The method of any one of the preceding embodiments, wherein said CO-Hb RBCs are type-O cells that are negative for the surface antigens Js^{a}, Kp^{a}, Wr^{a}, Di^{a}, V_{w}, V, Lu^{a} and C^{w}.

Embodiment 19. The method of any one of the preceding embodiments, wherein said CO-Hb RBCs are type-O cells that are positive for the surface antigens D, C, and e and having the Rh haplotype R₁ or are positive for the surface antigens D, c, and e and having the Rh haplotype R₂.

Embodiment 20. The method of any one of the preceding embodiments, wherein said CO-Hb RBCs are type-O cells that are positive for the surface antigens Lu^{b}, Js^{b}, Kp^{b}, and Yt^{a}.

Embodiment 21. The method of any one of the preceding embodiments, wherein said CO-Hb RBCs are type-O cells that are negative for the surface antigens Js^{a}, Kp^{a}, Wr^{a}, Di^{a}, V_{w}, V, Lu^{a} and C^{w}.

Embodiment 22. The method of any one of the preceding embodiments, wherein said CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a}.

Embodiment 23. The method of any one of the preceding embodiments, wherein said CO-Hb RBCs are type-O cells that are positive for binding of antibodies to D, C, E, c, e, f, Jk^{a}, Jk^{b}, Le^{a}, Le^{b}, P₁, I, IH, Vel, PP₁P^{k} and P antigens.

Embodiment 24. The method of any one of the preceding embodiments, wherein said CO-Hb RBCs are type-O cells having reduced or absent binding of antibodies to Fy^{a}, Fy^{b}, S, s, M, N, Xg^{a}, Pr, Ch^{a}, Rg^{a}, and Yk^{a}.

Embodiment 25. The method of any one of the preceding embodiments, wherein said CO-Hb RBCs are type-O cells that are positive for binding of antibodies to the D antigen and said CO-Hb RBCs are sensitized with anti-D(Rh₀) serum.

Embodiment 26. The method of any one of the preceding embodiments, wherein said CO-Hb RBCs are type-A cells that are positive for binding of antibodies to the A2 antigen.

Embodiment 27. The method of any one of the preceding embodiments, wherein said CO-Hb RBCs are type-B cells that are positive for binding of antibodies to the B antigen and are negative for binding of anti-D(Rh₀) antibodies.

Embodiment 28. The method of any one of the preceding embodiments, wherein said CO-Hb RBCs are type-A cells that are positive for binding of antibodies to the A₁ antigen and are negative for binding of anti-D(Rh₀) antibodies.

Embodiment 29. The method of any one of the preceding embodiments, wherein said CO-Hb RBCs are type-AB cells that are positive for binding of antibodies to the A₁, B antigens and the Rh antigens d, c, and e of Rh blood group rr (dce/dec).

Embodiment 30. The method of any one of the preceding embodiments, wherein said CO-Hb RBCs are type-O cells that are positive for binding of antibodies to the Rh antigens D, d, C, c, and e and having the Rh phenotype R₁r (DCe/dce).

Embodiment 31. The method of any one of the preceding embodiments, wherein said CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b}.

Embodiment 32. The method of any one of the preceding embodiments, wherein said CO-Hb RBCs are type-O cells that are positive for binding of antibodies to the Lu^{b}, Js^{b}, Kp^{b}, and Yt^{a} antigens.

Embodiment 33. The method of any one of the preceding embodiments, wherein said CO-Hb RBCs are type-O cells that are negative for the binding of antibodies to the Js^{a}, Kp^{a}, Wr^{a}, Di^{a}, V_{w}, V, Lu^{a} and C^{w} antigens.

Embodiment 34. The method of any one of the preceding embodiments, wherein said CO-Hb RBCs are type-O cells no profile in Resolve® Panel A instructions.

Embodiment 35. The method of any one of the preceding embodiments, wherein said CO-Hb RBCs are type-O cells no profile in Resolve® Panel B instructions.

Embodiment 36. The method of any one of the preceding embodiments, wherein said CO-Hb RBCs are type cells no profile in Resolve® Panel C instructions.

Embodiment 37. A kit comprising:
a) one or more vials of carbon monoxide saturated RBCs (CO-Hb RBCs), said vials comprising a buffer; and a plurality of CO-Hb RBCs having a common set of surface antigens selected from the group consisting of:
(i) CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
(ii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
(iii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w};
(iv) CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b};
(v) CO-Hb RBCs are type-O cells that are positive for the Rh antigens D, C, and e;
(vi) CO-Hb RBCs are type-O cells that are positive for the Rh antigens D, C, and e, I, Lu^{b}, Js^{b}, Kp^{b}, and Yt^{a};
(vii) CO-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w};
(viii) CO-Hb RBCs are type-A cells that are positive for the surface antigen A1;
(ix) CO-Hb RBCs are type-A cells that are positive for the surface antigen A1 and are negative for surface antigens D, C, and E;
(x) CO-Hb RBCs are type-A cells that are positive for the surface antigen A2;
(xi) CO-Hb RBCs are type-A cells that are positive for the surface antigen A2; and are negative for surface antigens D, C, and E;
(xii) CO-Hb RBCs are type-B cells that are positive for the surface antigen B;
(xiii) CO-Hb RBCs are type-B cells that are positive for the surface antigen B and are negative for surface antigens D, C, and E;
(xiv) CO-Hb RBCs are type-O cells that are positive for the surface antigens D, C, and e and having the Rh phenotype R₁R₁;
(xv) CO-Hb RBCs are type-O cells that are positive for the surface antigens D, C^{w}, and e and having the Rh phenotype R₁^{w}R₁;
(xvi) CO-Hb RBCs are type-O cells that are positive for the surface antigens D, c, and E and having the Rh phenotype R₂R₂;
(xvii) CO-Hb RBCs are type-O cells that are positive for the surface antigens d, c, and e and having the Rh phenotype rr;
(xviii) CO-Hb RBCs are type-O cells that are positive for the surface antigens D, C, and e and having the Rh phenotype R₁R₁ and are positive for the surface antigens Lu^{b}, Js^{b}, Kp^{b}, and Yt^{a};
(xix) CO-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a};
(xx) CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a};
(xxi) CO-Hb RBCs are type-O cells that are positive for the surface antigens d, c, and e and having the Rh phenotype rr and are positive for the surface antigens Lu^{b}, Js^{b}, Kp^{b}, and Yt^{a};
(xxii) CO-Hb RBCs are type-O cells that are positive for the surface antigens D, C, and e and having the Rh phenotype R₁R₁ and are negative for the surface antigens Js^{a}, Kp^{a}, Wr^{a}, Di^{a}, V_{w}, V, Lu^{a} and C^{w};
(xxiii) CO-Hb RBCs are type-O cells that are positive for the surface antigens D, C^{w}, and e and having the Rh phenotype R₁^{w}R₁ and are negative for the surface antigens Js^{a}, Kp^{a}, Wr^{a}, Di^{a}, V_{w}, V, Lu^{a} and C^{w};
(xxiv) CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, V_{w}, V, Lu^{a} and C^{w};
(xxv) CO-Hb RBCs are type-O cells that are positive for the surface antigens d, c, and e and having the Rh phenotype rr and are negative for the surface antigens Js^{a}, Kp^{a}, Wr^{a}, Di^{a}, V_{w}, V, Lu^{a} and C^{w};
(xxvi) CO-Hb RBCs are type-O cells that are positive for the surface antigens D, C, and e and having the Rh haplotype R₁ or are positive for the surface antigens D, c, and e and having the Rh haplotype R₂;
(xxvii) CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};
(xxviii)CO-Hb RBCs are type-O cells that are positive for the surface antigens D, C, and e and having the Rh haplotype R₁ and are negative for the surface antigens Js^{a}, Kp^{a}, Wr^{a}, Di^{a}, V_{w}, V, Lu^{a} and C^{w} or are positive for the surface antigens D, c, and e and having the Rh haplotype R₂ and are negative for the surface antigens Js^{a}, Kp^{a}, Wr^{a}, Di^{a}, V_{w}, V, Lu^{a} and C^{w};
(xxix) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a};
(xxx) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
(xxxi) CO-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
(xxxii) CO-Hb RBCs are type-O cells that are positive for binding of antibodies to the D antigen and said CO-Hb RBCs are sensitized with anti-D(Rh₀) serum; (xxxiii)CO-Hb RBCs are type-A cells that are positive for binding of antibodies to the A₂ antigen;
(xxxiv) CO-Hb RBCs are type-B cells that are positive for binding of antibodies to the B antigen and are negative for binding of anti-D(Rh₀) antibodies;
(xxxv) CO-Hb RBCs are type-A cells that are positive for binding of antibodies to the A₂ antigen and are negative for binding of anti-D(Rh₀) antibodies;
(xxxvi) CO-Hb RBCs are type-AB cells that are positive for binding of antibodies to the A₁, B antigens and the Rh antigens d, c, and e of Rh blood group rr (dce/dec);
(xxxvii) CO-Hb RBCs are type-O cells that are positive for binding of antibodies to the Rh antigens D, d, C, c, and e and having the Rh phenotype R₁r (DCe/dce);
(xxxviii) CO-Hb RBCs are type-O cells that are positive for binding of antibodies to the D (RH1), C (RH2), E (RH3), c (RH4), e (RH5), M, N, S, s, P₁, K, k, Fyₐ, Fy_{b}, Jkₐ, Jk^{b}, Le^{a} and Le^{b};
(xxxix) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jkₐ, Jk_{b}, Le^{a} and Le^{b} and are positive for binding of antibodies to the Lu^{b}, Js^{b}, Kp^{b}, and Yt^{a} antigens; and
(xl) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wᵣa, Di^{a}, Vw, V, Lu^{a} and C^{w} antigens; and b) instructions.

Embodiment 38. A vial of carbon monoxide saturated RBCs (CO-Hb RBCs) comprising a buffer and CO-Hb RBCs selected from the group consisting of:
(i) CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
(ii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
(iii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wr^{a}, Di^{a}, V^{w}, V, Lu^{a}, and C^{w};
(iv) CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b};
(v) CO-Hb RBCs are type-O cells that are positive for the Rh antigens D, C, and e;
(vi) CO-Hb RBCs are type-O cells that are positive for the Rh antigens D, C, and e, I, Lu^{b}, Js^{b}, Kp^{b}, and Yt^{a};
(vii) CO-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w};
(viii) CO-Hb RBCs are type-A cells that are positive for the surface antigen A1;
(ix) CO-Hb RBCs are type-A cells that are positive for the surface antigen A1 and are negative for surface antigens D, C, and E;
(x) CO-Hb RBCs are type-A cells that are positive for the surface antigen A2;
(xi) CO-Hb RBCs are type-A cells that are positive for the surface antigen A2; and are negative for surface antigens D, C, and E;
(xii) CO-Hb RBCs are type-B cells that are positive for the surface antigen B;
(xiii) CO-Hb RBCs are type-B cells that are positive for the surface antigen B and are negative for surface antigens D, C, and E;
(xiv) CO-Hb RBCs are type-O cells that are positive for the surface antigens D, C, and e and having the Rh phenotype RiRi;
(xv) CO-Hb RBCs are type-O cells that are positive for the surface antigens D, C^{w}, and e and having the Rh phenotype R₁^{w}R₁;
(xvi) CO-Hb RBCs are type-O cells that are positive for the surface antigens D, c, and E and having the Rh phenotype R₂R₂;
(xvii) CO-Hb RBCs are type-O cells that are positive for the surface antigens d, c, and e and having the Rh phenotype rr;
(xviii) CO-Hb RBCs are type-O cells that are positive for the surface antigens D, C, and e and having the Rh phenotype R₁R₁ and are positive for the surface antigens Lu^{b}, Js^{b}, Kp^{b}, and Yt^{a};
(xix) CO-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a};
(xx) CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a};
(xxi) CO-Hb RBCs are type-O cells that are positive for the surface antigens d, c, and e and having the Rh phenotype rr and are positive for the surface antigens Lu^{b}, Js^{b}, Kp^{b}, and Yt^{a};
(xxii) CO-Hb RBCs are type-O cells that are positive for the surface antigens D, C, and e and having the Rh phenotype R₁R₁ and are negative for the surface antigens Js^{a}, Kp^{a}, Wr^{a}, Di^{a}, V_{w}, V, Lu^{a} and C^{w};
(xxiii) CO-Hb RBCs are type-O cells that are positive for the surface antigens D, C^{w}, and e and having the Rh phenotype R₁^{w}R₁ and are negative for the surface antigens Js^{a}, Kp^{a}, Wr^{a}, Di^{a}, V_{w}, V, Lu^{a} and C^{w};
(xxiv) CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, V_{w}, V, Lu^{a} and C^{w};
(xxv) CO-Hb RBCs are type-O cells that are positive for the surface antigens d, c, and e and having the Rh phenotype rr and are negative for the surface antigens Js^{a}, Kp^{a}, Wr^{a}, Di^{a}, V_{w}, V, Lu^{a} and C^{w};
(xxvi) CO-Hb RBCs are type-O cells that are positive for the surface antigens D, C, and e and having the Rh haplotype R₁ or are positive for the surface antigens D, c, and e and having the Rh haplotype R₂;
(xxvii) CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};
(xxviii) CO-Hb RBCs are type-O cells that are positive for the surface antigens D, C, and e and having the Rh haplotype R₁ and are negative for the surface antigens Js^{a}, Kp^{a}, Wr^{a}, Di^{a}, V_{w}, V, Lu^{a} and C^{w} or are positive for the surface antigens D, c, and e and having the Rh haplotype R₂ and are negative for the surface antigens Js^{a}, Kp^{a}, Wr^{a}, Di^{a}, V_{w}, V, Lu^{a} and C^{w};
(xxix) CO-Hb RBCs are type-O cells that have been ficin treated and that are negative of the surface antigens M, N, Fy^{a}, Fy^{b}, S, s, Xg^{a}, Pr, Ch^{a}, R_{g}^{a} and Yk^{a};
(xxx) CO-Hb RBCs are type-O cells that have been ficin treated and that are negative of the surface antigens M, N, Fy^{a}, Fy^{b}, S, s, Xg^{a}, Pr, Ch^{a}, R_{g}^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
(xxxi) CO-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
(xxxii) CO-Hb RBCs are type-O cells that are positive for binding of antibodies to the D antigen and said cells are sensitized with anti-D(Rh₀) serum;
(xxxiii)CO-Hb RBCs are type-A cells that are positive for binding of antibodies to the A₂ antigen;
(xxxiv) CO-Hb RBCs are type-B cells that are positive for binding of antibodies to the B antigen and are negative for binding of anti-D(Rh₀) antibodies;
(xxxv) CO-Hb RBCs are type-A cells that are positive for binding of antibodies to the A₁ antigen and are negative for binding of anti-D(Rh₀) antibodies;
(xxxvi) CO-Hb RBCs are type-AB cells that are positive for binding of antibodies to the A₁, B antigens and the Rh antigens d, c, and e of Rh blood group rr (dce/dec);
(xxxvii) CO-Hb RBCs are type-O cells that are positive for binding of antibodies to the Rh antigens D, d, C, c, and e and having the Rh phenotype R₁r (DCe/dce);
(xxxviii) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b};
(xxxix) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and
(xl) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, Vw, V, Lu^{a} and C^{w} antigens.

Having now generally described the invention, the same will be more readily understood through reference to the following examples that are provided by way of illustration, and are not intended to be limiting of the present invention, unless specified.

Each periodical, patent, and other document or reference cited herein is herein incorporated by reference in its entirety.

### EXAMPLES

### Example 1

### Collection of Blood and Preparation of Red Cell Concentrate (RCC)

Blood for the preparation of Reagent RBCs is collected from an established donor into anti-coagulant solution using standard methods. Various known anticoagulants suitable for use in transfusion medicine are suitable including Citrate Phosphate Dextrose (CPD) and Acid Citrate Dextrose (ACD), but other anticoagulants such as ethylenediaminetetraacetic acid (EDTA) and ethylene glycol-bis(β-aminoethyl ether)-N,N,N′,N′-tetraacetic acid (EGTA) can be used as appropriate if the blood will not be used for transfusion. Collected blood is subjected to centrifugation or filtration to separate the white blood cells (WBC) and excess plasma to prepare packed red blood cells (pRBC) or Red Cell Concentrate (RCC).

### Example 2

### Preparation of Hb-CO Containing RCC (Hb-CO RCC)

Preferably without delay, the red cell concentrate (RCC) prepared in Example 1 is converted to Hb-CO by one of the following methods:

a. Rapid Gas Exchange:

RCC is held in a polyvinyl chloride or other suitable bag (150 ml or more) and carbon monoxide is introduced and the bag containing the RCC and CO are placed on a platelet shaker for about 10 minute. After incubation, the gas containing un-exchanged CO, released and residual oxygen, and carbon dioxide is expressed and replaced with fresh CO. After a second incubation of a platelet shaker for about 10 minutes, the gas is expressed a second time and replaced with a third volume of CO. Following a final incubation with shaking on a platelet shaker, the excess gas is removed and the Hb-CO RCC transferred under anaerobic conditions to suitable vials for further characterization, quality control, and storage.

b. Overnight Gas Exchange:
RCC is held in a polyvinyl chloride or other suitable bag (150 ml or more) and carbon monoxide is introduced and the bag containing the RCC and CO are placed on overnight at 4 ° C. with constant gentle shaking or at agitated at regular intervals (e.g., 3 to 5 x over 8 hours). After incubation, the CO containing excess gas is removed and the Hb-CO RCC transferred under anaerobic conditions to suitable vials for further characterization, quality control, and storage.

c. Membrane Gas Exchange

RCC held in a polyvinyl chloride or other suitable bag (150 ml or more) is pumped through a Sorin D100 oxygenator with carbon monoxide as the source gas. The RCC is pumped through the D100 using either a centrifugal or peristaltic pump for 30 minutes. Oxygen and CO levels are monitored until Hb-CO levels of greater than 95% are achieved.

d. Microbubble Gas Exchange:

RCC is held in a polyvinyl chloride or other suitable vented bag (150 ml or more) and carbon monoxide is bubbled through the RCC. Care is taken to ensure that the bubbles are no more than 1 μm in diameter to prevent lysis of the red blood cells. The resulting Hb-CO RCC is transferred under anaerobic conditions to suitable vials for further characterization, quality control, and storage.

e. Modified HEMANEXT® Oxygen Reduction Bag (ORB)

A HEMANEXT® Oxygen Reduction Bag (ORB) as described in U.S. Pat. No. 10,058,091, issued Aug. 28, 2018, is modified to remove the sorbent pack and the headspace filled with CO gas (100 to 200 ml). The CO containing ORB bag is agitated at room temperature on a platelet shaker for 30 minutes. Alternatively, the CO containing ORB bag is placed at 4° C. with either constant shaking or with agitation at regular intervals (e.g., 3 to 5× over 8 hours).

### Example 2

### Preparation and Packaging of HB-CO Reagent RBCs

Continue further manufacturing process with CO-treated RBC.

Package reagent RBC in a reagent bottle head and fill the head space CO under positive pressure and store at 4° C.

1. A method for preserving reagent red blood cells (RBC) comprising:
a) obtaining packed red blood cells;
b) flushing said packed red blood cells with a gas comprising carbon monoxide to prepare carbon monoxide saturated RBCs (CO-Hb RBCs); and
c) storing said CO-Hb RBCs under anaerobic conditions in the presence of carbon monoxide (CO), wherein surface antigens of said CO-Hb RBCs are stabilized.

2. The method of claim 1, wherein said gas does not comprise oxygen.

3. The method of claim 1, wherein said CO-Hb RBCs are blood group O cells that are positive for the surface antigens selected from the group consisting of D, C, c, E, e, CW, K, k, P₁, Fy^{a}, Fy_{b}, Jk^{a}, Jk^{b}, Le^{a}, Le^{b}, M, N, S, and s.

4. The method of claim 1, wherein said CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, N, S, s, Lu^{a}, and Lu^{b}.

5. The method of claim 1, wherein said CO-Hb RBCs are type-A cells that are positive for the surface antigen A2.

6. The method of claim 1, wherein said CO-Hb RBCs are type-B cells that are positive for the surface antigen B.

7. The method of claim 1, wherein said CO-Hb RBCs are type-O cells that
are positive for the surface antigens D, C, and e and having the Rh phenotype R₁R₁;
are positive for the surface antigens D, C^{w}, and e and having the Rh phenotype R₁^{w}R₁;
are positive for the surface antigens D, c, and E and having the Rh phenotype R₂R₂;
are positive for the surface antigens d, c, and e and having the Rh phenotype rr; or
Rh antigens D, C, and e.

8. The method of claim 7, wherein said CO-Hb RBCs are type-O cells that are positive for the surface antigens Lu^{b}, Js^{b}, Kp^{b}, and Yt^{a}; or positive for the surface antigens D, C, and e and having the Rh haplotype R₁ or are positive for the surface antigens D, c, and e and having the Rh haplotype R₂.

9. The method of claim 7, wherein said CO-Hb RBCs are type-O cells that are negative for the surface antigens Js^{a}, Kp^{a}, Wr^{a}, Di^{a}, Vw, V, Lu^{a} and C^{w}.

10. The method of claim 1, wherein said CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a}.

11. The method of claim 1, wherein said CO-Hb RBCs are type-O cells that are positive for binding of antibodies to the D antigen and said CO-Hb RBCs are sensitized with anti-D(Rh₀) serum.

12. The method of claim 1, wherein said CO-Hb RBCs are type-A cells that are positive for binding of antibodies to the A₂ antigen.

13. The method of claim 1, wherein said CO-Hb RBCs are type-B cells that are positive for binding of antibodies to the B antigen and are negative for binding of anti-D(Rh₀) antibodies.

14. The method of claim 1, wherein said CO-Hb RBCs are type-A cells that are positive for binding of antibodies to the Ai antigen and are negative for binding of anti-D(Rh₀) antibodies.

15. The method of claim 1, wherein said CO-Hb RBCs are type-AB cells that are positive for binding of antibodies to the Ai, B antigens and the Rh antigens d, c, and e of Rh blood group rr (dce/dec).

16. The method of claim 1, wherein said CO-Hb RBCs are type-O cells that are positive for binding of antibodies to the Rh antigens D, d, C, c, and e and having the Rh phenotype R₁r (DCe/dce); or
binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b}.

17. The method of claim 1, wherein said CO-Hb RBCs are type-O cells no profile in Resolve® Panel A instructions or Resolve® Panel B instructions.

18. The method of claim 1, wherein said CO-Hb RBCs are type-O cells no profile in Resolve® Panel C instructions.

19. A kit comprising:
a) one or more vials of carbon monoxide saturated RBCs (CO-Hb RBCs), said vials comprising a buffer; and
a plurality of CO-Hb RBCs having a common set of surface antigens selected from the group consisting of:
(i) CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
(ii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
(iii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a} and negative for the surface antigens Js^{a}, Kp^{a}, Wᵣa, Di^{a}, V^{w}, V, Lu^{a}, and C^{w};
(iv) CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b};
(v) CO-Hb RBCs are type-O cells that are positive for the Rh antigens D, C, and e;
(vi) CO-Hb RBCs are type-O cells that are positive for the Rh antigens D, C, and e, I, Lu^{b}, Js^{b}, Kp^{b}, and Yt^{a};
(vii) CO-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w};
(viii) CO-Hb RBCs are type-A cells that are positive for the surface antigen A1;
(ix) CO-Hb RBCs are type-A cells that are positive for the surface antigen A1 and are negative for surface antigens D, C, and E;
(x) CO-Hb RBCs are type-A cells that are positive for the surface antigen A2;
(xi) CO-Hb RBCs are type-A cells that are positive for the surface antigen A2; and are negative for surface antigens D, C, and E;
(xii) CO-Hb RBCs are type-B cells that are positive for the surface antigen B;
(xiii) CO-Hb RBCs are type-B cells that are positive for the surface antigen B and are negative for surface antigens D, C, and E;
(xiv) CO-Hb RBCs are type-O cells that are positive for the surface antigens D, C, and e and having the Rh phenotype R₁R₁;
(xv) CO-Hb RBCs are type-O cells that are positive for the surface antigens D, and e and having the Rh phenotype R₁^{w}Rₗ;
(xvi) CO-Hb RBCs are type-O cells that are positive for the surface antigens D, c, and E and having the Rh phenotype R₂R₂;
(xvii) CO-Hb RBCs are type-O cells that are positive for the surface antigens d, c, and e and having the Rh phenotype rr;
(xviii) CO-Hb RBCs are type-O cells that are positive for the surface antigens D, C, and e and having the Rh phenotype RiRi and are positive for the surface antigens Lu^{b}, Js^{b}, Kp^{b}, and Yt^{a};
(xix) CO-Hb RBCs are type-O cells that are positive for the surface antigens D, and e and having the Rh phenotype Rivaₗ and are positive for the surface antigens Lu^{b}, Js^{b}, Kp^{b}, and Yt^{a};
(xx) CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a};
(xxi) CO-Hb RBCs are type-O cells that are positive for the surface antigens d, c, and e and having the Rh phenotype R₁R₁ and are positive for the surface antigens Lu^{b}, Js^{b}, Kp^{b}, and Yt^{a};
(xxii) CO-Hb RBCs are type-O cells that are positive for the surface antigens D, C, and e and having the Rh phenotype R₁R₁ and are negative for the surface antigens Js^{a}, Kp^{a}, Wr^{a}, Di^{a}, Vw, V, Lu^{a} and C^{w};
(xxiii) CO-Hb RBCs are type-O cells that are positive for the surface antigens D, and e and having the Rh phenotype R₁^{w}R₁ and are negative for the surface antigens Js^{a}, Kp^{a}, Wr^{a}, Vw, V, Lu^{a} and C^{w};
(xxiv) CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, Vw, V, Lu^{a} and C^{w};
(xxv) CO-Hb RBCs are type-O cells that are positive for the surface antigens d, c, and e and having the Rh phenotype rr and are negative for the surface antigens Js^{a}, Kp^{a}, Wr^{a}, Di^{a}, Vw, V, Lu^{a} and C^{w};
(xxvi) CO-Hb RBCs are type-O cells that are positive for the surface antigens D, C, and e and having the Rh haplotype R₁ or are positive for the surface antigens D, c, and e and having the Rh haplotype R₂;
(xxvii) CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};
(xxviii) CO-Hb RBCs are type-O cells that are positive for the surface antigens D, C, and e and having the Rh haplotype R₁ and are negative for the surface antigens Js^{a}, Kp^{a}, Wr^{a}, Di^{a}, Vw, V, Lu^{a} and C^{w} or are positive for the surface antigens D, c, and e and having the Rh haplotype R₂ and are negative for the surface antigens Js^{a}, Kp^{a}, Wr^{a}, Di^{a}, Vw, V, Lu^{a} and C^{w};
(xxix) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a};
(xxx) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
(xxxi) CO-Hb RBCs are type-O cells that have been ficin treated and that are negative of the surface antigens M, N, Fy^{a}, Fy^{b}, S, s, Xg^{a}, Pr, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
(xxxii) CO-Hb RBCs are type-O cells that are positive for binding of antibodies to the D antigen and said CO-Hb RBCs are sensitized with anti-D(Rh₀) serum;
(xxxiii) CO-Hb RBCs are type-A cells that are positive for binding of antibodies to the A₂ antigen;
(xxxiv) CO-Hb RBCs are type-B cells that are positive for binding of antibodies to the B antigen and are negative for binding of anti-D(Rh₀) antibodies;
(xxxv) CO-Hb RBCs are type-A cells that are positive for binding of antibodies to the A₁ antigen and are negative for binding of anti-D(Rh₀) antibodies;
(xxxvi) CO-Hb RBCs are type-AB cells that are positive for binding of antibodies to the A₁, B antigens and the Rh antigens d, c, and e of Rh blood group rr (dce/dec);
(xxxvii) CO-Hb RBCs are type-O cells that are positive for binding of antibodies to the Rh antigens D, d, C, c, and e and having the Rh phenotype R₁r (DCe/dce);
(xxxviii) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b};
(xxxix) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and
(xl) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, Vw, V, Lu^{a} and C^{w} antigens; and
b) instructions.

20. A vial of carbon monoxide saturated RBCs (CO-Hb RBCs) comprising a buffer and CO-Hb RBCs selected from the group consisting of:
(i) CO-Hb RBCs that are blood group O cells and are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, and s;
(ii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, Kp^{b}, and Yt^{a};
(iii) CO-Hb RBCs are blood group O cells that are positive for the surface antigens selected from the group consisting of D, C, c, E, e, CW, K, k, P₁, Fy^{a}, Fy^{b}, Jk^{a}, Jk^{b}, Le^{a}, Le^{b}, M, N, S, s, I, Lu^{a}, Lu^{b}, Js^{b}, K_{b}, and Yt^{a} and negative for the surface antigens Js^{a}, Kp^{a}, Wᵣa, Di^{a}, V^{w}, V, Lu^{a}, and C^{w};
(iv) CO-Hb RBCs are type-O cells that are negative for the surface antigens D, C, c, E, e, f, CW, K, k, P₁, Fy^{a}, Fy^{b}, Jk^{a}, Jk^{b}, Le^{a}, Le^{b}, M, N, S, s, Lu^{a}, and Lu^{b};
(v) CO-Hb RBCs are type-O cells that are positive for the Rh antigens D, C, and e;
(vi) CO-Hb RBCs are type-O cells that are positive for the Rh antigens D, C, and e, I, Lu^{b}, Js^{b}, Kp^{b}, and Yt^{a};
(vii) CO-Hb RBCs are type-O cells that are positive for the Rh antigens D, C, and e, I, Lu^{b}, Js^{b}, Kp^{b}, and Yt^{a}, and that are negative for the surface antigens Js^{a}, Kp^{a}, Wr^{a}, Di^{a} V^{w}, V, Lu^{a} and C^{w};
(viii) CO-Hb RBCs are type-A cells that are positive for the surface antigen A1;
(ix) CO-Hb RBCs are type-A cells that are positive for the surface antigen A1 and are negative for surface antigens D, C, and E;
(x) CO-Hb RBCs are type-A cells that are positive for the surface antigen A2;
(xi) CO-Hb RBCs are type-A cells that are positive for the surface antigen A2; and are negative for surface antigens D, C, and E;
(xii) CO-Hb RBCs are type-B cells that are positive for the surface antigen B;
(xiii) CO-Hb RBCs are type-B cells that are positive for the surface antigen B and are negative for surface antigens D, C, and E;
(xiv) CO-Hb RBCs are type-O cells that are positive for the surface antigens D, C, and e and having the Rh phenotype R₁R₁;
(xv) CO-Hb RBCs are type-O cells that are positive for the surface antigens D, C^{w}, and e and having the Rh phenotype R₁^{w}R₁;
(xvi) CO-Hb RBCs are type-O cells that are positive for the surface antigens D, c, and E and having the Rh phenotype R₂R₂;
(xvii) CO-Hb RBCs are type-O cells that are positive for the surface antigens d, c, and e and having the Rh phenotype rr;
(xviii) CO-Hb RBCs are type-O cells that are positive for the surface antigens D, C, and e and having the Rh phenotype R₁R₁ and are positive for the surface antigens Lu^{b}, Js^{b}, Kp^{b}, and Yt^{a};
(xix) CO-Hb RBCs are type-O cells that are positive for the surface antigens D, C^{w}, and e and having the Rh phenotype R₁^{w}R₁ and are positive for the surface antigens Lu^{b}, Js^{b}, Kp^{b}, and Yt^{a};
(xx) CO-Hb RBCs are type-O cells that are positive for the surface antigens D, c, and E and having the Rh phenotype R₂R₂ and are positive for the surface antigens Lu^{b}, Js^{b}, Kp^{b}, and Yt^{a};
(xxi) CO-Hb RBCs are type-O cells that are positive for the surface antigens d, c, and e and having the Rh phenotype rr and are positive for the surface antigens Lu^{b}, Js^{b}, Kp^{b}, and Yt^{a};
(xxii) CO-Hb RBCs are type-O cells that are positive for the surface antigens D, C, and e and having the Rh phenotype RiRi and are negative for the surface antigens Js^{a}, Kp^{a}, Wr^{a}, Di^{a}, Vw, V, Lu^{a} and C^{w};
(xxiii) CO-Hb RBCs are type-O cells that are positive for the surface antigens D, C^{w}, and e and having the Rh phenotype R₁^{w}R₁ and are negative for the surface antigens Js^{a}, Kp^{a}Wr^{a}, Di^{a}, Vw, V, Lu^{a} and C^{w};
(xxiv) CO-Hb RBCs are type-O cells that are positive for the surface antigens D, c, and E and having the Rh phenotype R₂R₂ and are negative for the surface antigens Js^{a}, Kp^{a}, Wr^{a}, Di^{a}, Vw, V, Lu^{a} and C^{w};
(xxv) CO-Hb RBCs are type-O cells that are positive for the surface antigens d, c, and e and having the Rh phenotype rr and are negative for the surface antigens Js^{a}, Kp^{a}, Wr^{a}, Di^{a}, Vw, V, Lu^{a} and C^{w};
(xxvi) CO-Hb RBCs are type-O cells that are positive for the surface antigens D, C, and e and having the Rh haplotype R₁ or are positive for the surface antigens D, c, and e and having the Rh haplotype R₂;
(xxvii) CO-Hb RBCs are type-O cells that are positive for the surface antigens D, C, and e and having the Rh haplotype R₁ and are positive for the surface antigens Lu^{b}, Js^{b}, Kp^{b}, and Yt^{a} or are positive for the surface antigens D, c, and e and having the Rh haplotype R₂ CO-Hb RBCs and are positive for the surface antigens Lu^{b}, Js^{b}, Kp^{b}, and Yt^{a};
(xxviii) CO-Hb RBCs are type-O cells that are positive for the surface antigens D, C, and e and having the Rh haplotype Rᵢ and are negative for the surface antigens Js^{a}, Kp^{a}, Wr^{a}, Di^{a}, Vw, V, Lu^{a} and C^{w} or are positive for the surface antigens D, c, and e and having the Rh haplotype R₂ and are negative for the surface antigens Js^{a}, Kp^{a}, Wr^{a}, Di^{a}, Vw, V, Lu^{a} and C^{w};
(xxix) CO-Hb RBCs are type-O cells that have been ficin treated and that are negative of the surface antigens M, N, Fy^{a}, Fy^{b}, S, s, Xg^{a}, Pr, Ch^{a}, Rg^{a} and Yk^{a}.
(xxx) CO-Hb RBCs are type-O cells that have been ficin treated and that are negative of the surface antigens M, N, Fy^{a}, Fy^{b}, S, s, Xg^{a}, Pr^{a}, Ch^{a}, Rg^{a} and Yk^{a} and are positive for binding of antibodies to D, C, E, c, e, f, Jk^{a}, Jk^{b}, Le^{a}, Le^{b}, P₁, I, IH, Vel, PP₁P^{k} and P antigens;
(xxxi) CO-Hb RBCs are type-O cells that have been ficin treated and that are negative of the surface antigens M, N, Fy^{a}, Fy^{b}, S, s, Xg^{a}, Pr^{a}, Ch^{a}, Rg^{a} and having reduced or absent binding of antibodies to Fy^{a}, Fy^{b}, S, s, M, N, Xg^{a}, Pr, Ch^{a}, Rg^{a}, and Yk^{a};
(xxxii) CO-Hb RBCs are type-O cells that are positive for binding of antibodies to the D antigen and said cells are sensitized with anti-D(Rh₀) serum;
(xxxiii) CO-Hb RBCs are type-A cells that are positive for binding of antibodies to the A₂ antigen;
(xxxiv) CO-Hb RBCs are type-B cells that are positive for binding of antibodies to the B antigen and are negative for binding of anti-D(Rh₀) antibodies;
(xxxv) CO-Hb RBCs are type-A cells that are positive for binding of antibodies to the Ai antigen and are negative for binding of anti-D(Rh₀) antibodies;
(xxxvi) CO-Hb RBCs are type-AB cells that are positive for binding of antibodies to the A₁, B antigens and the Rh antigens d, c, and e of Rh blood group rr (dce/dec);
(xxxvii) CO-Hb RBCs are type-O cells that are positive for binding of antibodies to the Rh antigens D, d, C, c, and e and having the Rh phenotype R₁r (DCe/dce);
(xxxviii) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b};
(xxxix) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are positive for binding of antibodies to the Lu^{b}, Js^{b}, Kp^{b}, and Yt^{a} antigens; and
(xl) CO-Hb RBCs are type-O cells that are positive for binding of antibodies to the D (RH1), C (RH2), E (RH3), c (RH4), e (RH5), M, N, S, s, P₁, K, k, Fy^{a}, Fy^{b}, Jk^{a}, Jk^{b}, Le^{a} and Le^{b} and are negative for the binding of antibodies to the Js^{a}, Kp^{a}, Wr^{a}, Di^{a}, Vw, V, Lu^{a} and C^{w} antigens.

21. (canceled)
